(19) United States
(12) Patent Application Publication
(10) Pub. No.: US 2022/0273747 A1
(43) Pub. Date: Sep. 1, 2022

(54) NATURAL ESSENTIAL OIL COMPOSITION FOR RELAXING NERVES, SLEEPING WELL AND PREVENTING COLD AND FLU

(71) Applicant: SUNSTAR JOINT STOCK COMPANY, Hanoi (VN)

(72) Inventor: Thi Huong Lien NGUYEN, Hanoi (VN)

(21) Appl. No.: 17/638,052

(22) PCT Filed: Jul. 20, 2020
(86) PCT No.: PCT/VN2020/000006

§ 371 Date: Feb. 24, 2022

(30) Foreign Application Priority Data
Aug. 29, 2019 (VN) 1-2019-04787

(51) Int. Cl. A61K 36/53; A61K 36/534; A61K 36/23; A61K 36/61; A61K 36/752; A61K 36/899; A61K 36/282

(52) U.S. Cl. CPC A61K 36/53; A61K 36/534; A61K 36/23; A61K 36/61; A61K 36/752; A61K 36/899; A61K 36/282

(57) ABSTRACT

The present invention provides a natural essential oil composition for relaxing nerves, sleeping well and preventing cold and flu. The composition comprises holy basil, lemongrass, patchouli, old coriander seeds, and tangerine peels, wherein the amount (wt %} of holy basil essential oil is about: 0.1% to about 60%; the amount (wt %) of lemongrass essential oil is about 0.1% to about 55; the amount (wt %) of patchouli essential oil is about 0.1% to about 30%; the amount: (wt %) of essential oil of old coriander seeds is about 0.1 to about 50% and the amount (wt %) of essential oil of tangerine peels is about 0.1% to about 50%. The composition of the present invention further comprises additional essential oils for further use purposes. The composition also comprises carrier oils and solvents for adjusting suit able concentrations of the composition.

### FIELD OF INVENTION

The present invention relates to a natural essential oil composition for relaxing nerves, sleeping well, preventing cold and flu, and other use purposes.

### DESCRIPTION OF PRIOR ART OF THE PRESENT INVENTION

Vegetable oils are generally known to have powerful effects on the brain and nervous system through the olfactory nerves. Each essential oil usually contains about 100 chemicals that may affect the entire body of human. Essential oils work on the body and mind, helping to relax, reduce fatigue, anxiety, thereby helping easily falling asleep and deeply sleeping well. Aromatherapy has been appeared for thousands of years. For years, scientists have been conducting research on the benefits of sleep-promoting, stress-relieving, analgesic and mood-regulating caused by essential oils.

The essential oils helpful for sleep are divided into two main types: soothing moods, reducing anxiety, and reducing nasal congestion, reducing snoring. According to researches, using essential oils before sleep can improve sleep quality without causing any side effects like using sleeping pills.

The essential oils suitable for bedrooms are usually the ones having warm and sweet scent to help body relax, sedate and easier of falling asleep. A study published in the British medical journal Lancet found that elderly patients would sleep well when the essential oils of lavender, sandalwood, rose, etc. were put in their bedrooms at night. The elderly patients belong to a group of often having trouble falling asleep and need to take sleeping pills.

It has been known that many essential oils have effects on relaxation, sedation, sleeping well such as holy basil essential oil (Ocimum gratissimum), lemongrass essential oil (Cymbopogon citratus—Lemon grass), patchouli essential oil (Pogostemon cablin—Patchouli), essential oil of old coriander seeds (Coriandrum sativum fruit), tea tree essential oil (Oleum cajeputi), essential oil of tangerine peels (Citrus deliciosa peel), essential oil of grapefruit peels (Citrus grandis (Pomelo) peel), essential oil of orange peels (Citrus aurantium peel), mint essential oil (Mentha arvensis), garlic essential oil (Allium sativum), anise essential oil (Illicium verum fruit), mugwort essential oil (Artemisia vulgaris), dill essential oil (Anethum graveolens), camphor essential oil (Cinnamomum camphora), lemon basil essential oil (Plectranthus amboinicus), perilla essential oil (Perilla frutescens), oregano essential oil (Elsholtzia ciliate), rose essential oil (Rosa chinensis), maychang essential oil (Litsea cubeba), eucalyptus essential oil (Eucalyptus sp.), clove essential oil (Syzygium aromaticum), turmeric essential oil (Cucurma longa), ylang ylang essential oil (Cananga odorata), sarsi essential oils (Smilax regelii), eucalyptus essential oils (Eucalyptus globules), Cupressus funebris essential oils (Cupressus funebris), etc.

These essential oils are often used separately, by applying on the skin, or steaming the room, or spraying into the bedroom to give a feeling of relaxation, sleeping well. In addition to the effects mentioned above, these essential oils also have anti-inflammatory, insect repellent, and mosquito repellent effects, as well as pleasant aroma.

Practically, the essential oils are not only used separately, but can also be used as a combination of many essential oils together, thus creating a variety of mixtures of essential oils with different effects and aromas, suitable for variety of different uses.

Korean patent publication No. KR20110089169A disclosed a mixture of essential oils for applying on skin that comprises essential oils of tangerine peels, lavender essential oils, and Roman chamomile essential oils, that have effects of relaxing nerves.

WO2014/112676 A1 disclosed a mixture of the essential oils for massaging on body skin to make deep sleep that comprises valerian essential oils, lavender essential oils and Roman chamomile essential oils.

However, there are still needs of variety of essential oil compositions that not only provide effects of comfort, relaxation, sleeping well but also effects of deodorization, disinfection, flu prevention, hair growth stimulation, insect repellent, etc., made from a variety of different essential oil ingredients thus ensuring a stable source of raw materials for the production of the essential oil compositions.

### SUMMARY OF THE INVENTION

The purpose of the present invention is to provide a natural essential oil composition which not only helps to bring comfort, relaxation, and sleeping well, but also has the effects of deodorization, disinfection, flu prevention, hair growth stimulation, insect repellent, etc., made from a variety of different essential oil ingredients.

The present invention will be described below. It should be noted that all the amounts here are presented in percentage by weight (wt %).

In the first aspect, the present invention provides a natural essential oil composition for releasing aroma and for relaxing nerves, sleeping well and preventing cold and flu, said natural essential oil composition comprises ingredients of essential oils from herbs selected from the group consisting of: Holy basil, Lemongrass, Patchouli, old coriander seeds, and tangerine peels. In one preferred embodiment, the natural essential oil composition comprises with the amounts of percentage by weight (wt %): holy basil essential oil is about 0.1% to about 60%; lemongrass essential oil is about 0.1% to about 55; patchouli essential oil is about 0.1% to about 30%; essential oil of old coriander seeds is about 0.1 to about 50% and essential oil of tangerine peels is about 0.1% to about 50%.

Preferably, the natural essential oil composition comprises with the amounts of percentage by weight (wt %): holy basil essential oil is about 0.1% to about 45; lemongrass essential oil is about 0.1% to about 30%; patchouli essential oil is about 0.1% to about 20%; essential oil of old coriander seeds is about 0.1 to about 40% and essential oil of tangerine peels is about 0.1% to about 30%.

More preferably, the natural essential oil composition comprises with the amounts of percentage by weight (wt %): holy basil essential oil is about 0.1% to about 30%; lemongrass essential oil is about 0.1% to about 25%; patchouli essential oil is about 0.1% to about 15; essential oil of old coriander seeds is about 0.1 to about 30% and essential oil of tangerine peels is about 0.1% to about 25%.

The natural essential oil composition according to the present invention have many different effects as a combination of the effects of each essential oil ingredient such as: relaxing nerves, sleeping quickly and well, preventing cold and flu, anti-fatigue, deodorization, enhancing immunity, stimulating hair growth, treating acne, whitening skin, etc.

In addition to the above-mentioned basic ingredients, the composition of the present invention may further comprises other ingredients that have the same effects as the above ingredients. Specifically, in another embodiment, the composition of invention may further comprises additional ingredients: tea tree oil; grapefruit peel oil; and orange peel oil.

Preferably, said additional ingredients have their amounts of percentage by weight (wt %) as follows: tea tree essential oil is about 0% to about 60%; essential oil of grapefruit peels is about 0% to about 50%; and essential oil of orange peels is about 0% to about 50. More preferably, said additional ingredients have their amounts of percentage by weight (wt %) as follows: tea tree essential oil is about 0% to about 40%; essential oil of grapefruit peels is about 0% to about 35; and essential oil of orange peels is about 0% to about 30%. Most preferably, said additional ingredients have their amounts of percentage by weight (wt %) as follows: tea tree essential oil is about 0% to about 30%; essential oil of grapefruit peels is about 0% to about 20%; and essential oil of orange peels is about 0% to about 25%.

The addition of the above mentioned oils makes the composition of the invention more effective in stimulating hair growth and create a sweet scent, in addition to the effects of the mixture listed above.

In still another embodiment, said additional ingredients may comprises at least one of essential oils selected from the group of: mint is about 0% to about 50%; garlic essential oil is about 0% to about 30%; anise essential oil is about 0% to about 40%; mugwort essential oil is about 0% to about 50%; dill essential oil is about 0% to about 50%; camphor essential oil is about 0% to about 30%; basil essential oil is about 0% to about 30%; lavender essential oil is about 0% to about 30%; perilla essential oil is about 0% to about 50%; and oregano essential oil is about 0% to about 50. Preferably, said additional ingredients have content ratios as follows: mint essential oil is about 0% to about 40%; garlic essential oil is about 0% to about 20%; anise essential oil is about 0% to about 25%; mugwort essential oil is about 0% to about 40%; dill essential oil is about 0% to about 40%; camphor essential oil is about 0% to about 25%; basil essential oil is about 0% to about 25%; lavender essential oil is about 0% to about 25%; perilla essential oil is about 0% to about 40%; and oregano essential oil is about 0% to about 40. Most preferably, said additional ingredients have content ratios as follows: mint essential oil is about 0% to about 30%; garlic essential oil is about 0% to about 10%; anise essential oil is about 0% to about 15; mugwort essential oil is about 0% to about 30%; dill essential oil is about 0% to about 30%; camphor essential oil is about 0% to about 20%; basil essential oil is about 0% to about 20%; lavender essential oil is about 0% to about 20%; perilla essential oil is about 0% to about 30%; and oregano essential oil is about 0% to about 30%.

The addition of the above mentioned oils makes the composition according to the above said embodiment have additional effects such as supporting treatment of respiratory diseases, reducing pain, stimulating digestion, blood circulation, and treating coughs.

In yet another embodiment, the composition of the present invention further comprises the following ingredients: frankincense essential oil is about 0% to about 20%; mint essential oil is about 0% to about 50%; garlic essential oil is about 0% to about 30%; anise essential oil is about 0% to about 40%; mugwort essential oil is about 0% to about 50%; dill essential oil is about 0% to about 50%; camphor essential oil is about 0% to about 30%; cajeput essential oil is about 0% to about 50%; ginger essential oil is about 0% to about 40%; jasmine essential oil is about 0% to about 50%; lemon basil essential oil is about 0% to about 30%; lavender essential oil is about 0% to about 30%; perilla essential oil is about 0% to about 50%; oregano essential oil is about 0% to about 50%; rose essential oil is about 0% to about 50%; maychang essential oil is about 0% to about 40%; pomu essential oil is about 0% to about 50%; eucalyptus essential oil is about 0% to about 30%; clove essential oil is about 0% to about 40%; turmeric essential oil is about 0% to about 50%; green tea essential oil is about 0% to about 50%; ylang ylang essential oil is about 0% to about 50%; sarsi essential oils is about 0% to about 50%; eucalyptus essential oils is about 0% to about 30%; and Cupressus funebris essential oils is about 0% to about 20%. Preferably, the composition of the present invention further comprises: frankincense essential oil is about 0% to about 15; mint essential oil is about 0% to about 40%; garlic essential oil is about 0% to about 20%; anise essential oil is about 0% to about 25%; mugwort essential oil is about 0% to about 40%; dill essential oil is about 0% to about 40%; camphor essential oil is about 0% to about 25%; cajeput essential oil is about 0% to about 40%; ginger essential oil is about 0% to about 30%; jasmine essential oil is about 0% to about 40%; lemon basil essential oil is about 0% to about 25%; lavender essential oil is about 0% to about 25%; perilla essential oil is about 0% to about 40%; oregano essential oil is about 0% to about 40%; rose essential oil is about 0% to about 40%; maychang essential oil is about 0% to about 30%; pomu essential oil is about 0% to about 40%; eucalyptus essential oil is about 0% to about 25%; clove essential oil is about 0% to about 30%; turmeric essential oil is about 0% to about 40%; green tea essential oil is about 0% to about 40%; ylang ylang essential oil is about 0% to about 40%; sarsi essential oils is about 0% to about 40%; eucalyptus essential oils is about 0% to about 25%; and Cupressus funebris essential oils is about 0% to about 15%. Most preferably, the composition further comprises: frankincense essential oil is about 0% to about 10%; mint essential oil is about 0% to about 30%; garlic essential oil is about 0% to about 10%; anise essential oil is about 0% to about 15; mugwort essential oil is about 0% to about 30%; dill essential oil is about 0% to about 30%; camphor essential oil is about 0% to about 20%; cajeput essential oil is about 0% to about 30%; ginger essential oil is about 0% to about 20%; jasmine essential oil is about 0% to about 30%; lemon basil essential oil is about 0% to about 20%; lavender essential oil is about 0% to about 20%; perilla essential oil is about 0% to about 30%; oregano essential oil is about 0% to about 30%; rose essential oil is about 0% to about 30%; maychang essential oil is about 0% to about 20%; pomu essential oil is about 0% to about 30%; eucalyptus essential oil is about 0% to about 20%; clove essential oil is about 0% to about 20%; turmeric essential oil is about 0% to about 30%; green tea essential oil is about 0% to about 30%; ylang ylang essential oil is about 0% to about 30%; sarsi essential oils is about 0% to about 30%; eucalyptus essential oils is about 0% to about 20%; and Cupressus funebris essential oils is about 0% to about 10%.

The addition of the above mentioned oils makes the composition according to the above said embodiment have additional effects such as fixing aroma, reducing nasal congestion, treating bone and joint pains, strengthening muscles, preventing hyperemia, preventing oxidation, reducing blood pressure, especially increasing libido.

In the second aspect, the present invention provides a composition comprising natural essential oils as mentioned above, wherein in order to facilitate the adjustment of the concentration of the mixture of essential oils to suit the required needs, the composition further comprises carrier oil, solvent with appropriate proportion. The carrier oil has a nourishing, soothing effect when used on skin. In one preferred embodiment, said carrier oil is selected from at least one of the group consisting of coconut oil, argan oil, olive oil, almond oil, jojoba oil, vanilla oil, neem oil, each is about 0% to about 70%; ethylic alcohol of about 0% to about 20 and water of about 0% to about 15%. Preferably, said carrier oil ingredients have the following ratios: coconut oil, argan oil, olive oil, almond oil, jojoba oil, vanilla oil, neem oil, each of about 0% to about 50%; ethylic alcohol of about 0% to about 10 and water of about 0% to about 10%. Most preferably, said carrier oil ingredients have the following ratios: coconut oil, argan oil, olive oil, almond oil, jojoba oil, vanilla oil, neem oil each of about 0% to about 30%; ethylic alcohol of about 0% to about 8% and water of about 0% to about 8%.

The composition of the present invention is prepared in the usual method, by mixing the essential oil ingredients in the proportions as mentioned above (to obtain a mixture of the essential oils of the present invention), then mixing them with the carrier oils in the proportions as mentioned above (to obtain the composition of the present invention). The mixing temperature is not over 25° C.

### DETAILED DESCRIPTION

Hereafter, the present invention will be described in details. It should be noted that all the amounts here are presented in percentage by weight (wt %). However, the invention is not intended to be limited to the specific terms so selected, and it is to be understood that each specific term includes all technical equivalents which operate in a similar manner to accomplish a similar purpose.

In the first aspect, the present invention provides a natural essential oil composition for relaxing nerves, sleeping well and preventing cold and flu. Said composition comprises ingredients of essential oils from herbs selected from the group consisting of: holy basil, lemongrass, patchouli, old coriander seeds, and tangerine peels. These ingredients are presented in wt % as follows: holy basil essential oil is about 0.1% to about 60%; lemongrass essential oil is about 0.1% to about 55; patchouli essential oil is about 0.1% to about 30%; essential oil of old coriander seeds is about 0.1 to about 50% and essential oil of tangerine peels is about 0.1% to about 50%.

Preferably, the natural essential oil composition comprises from about 0.1% to about 45% of holy basil essential oil; from about 0.1% to about 30% of lemongrass essential oil; from about 0.1% to about 20% of patchouli essential oil; form about 0.1 to about 40% of essential oil of old coriander seeds and from about 0.1% to about 30% of essential oil of tangerine peels.

Most preferably, the natural essential oil composition comprises from about 0.1% to about 30% of holy basil essential oil; from about 0.1% to about 25 of lemongrass essential oil; from about 0.1% to about 15 of patchouli essential oil; from about 0.1 to about 30% of essential oil of old coriander seeds and from about 0.1% to about 25% of essential oil of tangerine peels.

In a preferred embodiment, the composition of the present invention further comprises tea tree essential oil, essential oil of grapefruit peels, and essential oil of orange peels, additionally.

Preferably, these additional ingredients are presented in wt % as follows: tea tree essential oil is about 0% to about 60%; essential oil of grapefruit peels is about 0% to about 50%; and essential oil of orange peels is about 0% to about 50. More preferably, said additional ingredients have their amounts of percentage by weight (wt %) as follows: tea tree essential oil is about 0% to about 40%; essential oil of grapefruit peels is about 0% to about 35; and essential oil of orange peels is about 0% to about 30%. Most preferably, said additional ingredients have their amounts of percentage by weight (wt %) as follows: tea tree essential oil is about 0% to about 30%; essential oil of grapefruit peels is about 0% to about 20%; and essential oil of orange peels is about 0% to about 25%.

In another embodiment, said additional ingredients in the composition may further comprises at least one of mint essential oil is about 0% to about 50%; garlic essential oil is about 0% to about 30%; anise essential oil is about 0% to about 40%; mugwort essential oil is about 0% to about 50%; dill essential oil is about 0% to about 50%; camphor essential oil is about 0% to about 30%; basil essential oil is about 0% to about 30%; lavender essential oil is about 0% to about 30%; perilla essential oil is about 0% to about 50%; and oregano essential oil is about 0% to about 50. Preferably, said additional ingredients have content ratios as follows: mint essential oil is about 0% to about 40%; garlic essential oil is about 0% to about 20%; anise essential oil is about 0% to about 25%; mugwort essential oil is about 0% to about 40%; dill essential oil is about 0% to about 40%; camphor essential oil is about 0% to about 25%; basil essential oil is about 0% to about 25%; lavender essential oil is about 0% to about 25%; perilla essential oil is about 0% to about 40%; and oregano essential oil is about 0% to about 40. Most preferably, said additional ingredients have content ratios as follows: mint essential oil is about 0% to about 30%; garlic essential oil is about 0% to about 10%; anise essential oil is about 0% to about 15; mugwort essential oil is about 0% to about 30%; dill essential oil is about 0% to about 30%; camphor essential oil is about 0% to about 20%; basil essential oil is about 0% to about 20%; lavender essential oil is about 0% to about 20%; perilla essential oil is about 0% to about 30%; and oregano essential oil is about 0% to about 30%.

In still further embodiment, the composition of the present invention further comprises frankincense essential oil is about 0% to about 20%; mint essential oil is about 0% to about 50%; garlic essential oil is about 0% to about 30%; anise essential oil is about 0% to about 40%; mugwort essential oil is about 0% to about 50%; dill essential oil is about 0% to about 50%; camphor essential oil is about 0% to about 30%; cajeput essential oil is about 0% to about 50%; ginger essential oil is about 0% to about 40%; jasmine essential oil is about 0% to about 50%; lemon basil essential oil is about 0% to about 30%; lavender essential oil is about 0% to about 30%; perilla essential oil is about 0% to about 50%; oregano essential oil is about 0% to about 50%; rose essential oil is about 0% to about 50%; maychang essential oil is about 0% to about 40%; pomu essential oil is about 0% to about 50%; eucalyptus essential oil is about 0% to about 30%; clove essential oil is about 0% to about 40%; turmeric essential oil is about 0% to about 50%; green tea essential oil is about 0% to about 50%; ylang ylang essential oil is about 0% to about 50%; sarsi essential oils is about 0% to about 50%; eucalyptus essential oils is about 0% to about 30%; and Cupressus funebris essential oils is about 0% to about 20%. Preferably, the composition of the present invention further comprises ingredients with their content ratios as follows: frankincense essential oil is about 0% to about 15; mint essential oil is about 0% to about 40%; garlic essential oil is about 0% to about 20%; anise essential oil is about 0% to about 25%; mugwort essential oil is about 0% to about 40%; dill essential oil is about 0% to about 40%; camphor essential oil is about 0% to about 25%; cajeput essential oil is about 0% to about 40%; ginger essential oil is about 0% to about 30%; jasmine essential oil is about 0% to about 40%; lemon basil essential oil is about 0% to about 25%; lavender essential oil is about 0% to about 25%; perilla essential oil is about 0% to about 40%; oregano essential oil is about 0% to about 40%; rose essential oil is about 0% to about 40%; maychang essential oil is about 0% to about 30%; pomu essential oil is about 0% to about 40%; eucalyptus essential oil is about 0% to about 25%; clove essential oil is about 0% to about 30%; turmeric essential oil is about 0% to about 40%; green tea essential oil is about 0% to about 40%; ylang ylang essential oil is about 0% to about 40%; sarsi essential oils is about 0% to about 40%; eucalyptus essential oils is about 0% to about 25%; and Cupressus funebris essential oils is about 0% to about 15%. Most preferably, the composition further comprises các ingredient vói tŷ lȩ̂ sau: frankincense essential oil is about 0% to about 10%; mint essential oil is about 0% to about 30%; garlic essential oil is about 0% to about 10%; anise essential oil is about 0% to about 15; mugwort essential oil is about 0% to about 30%; dill essential oil is about 0% to about 30%; camphor essential oil is about 0% to about 20%; cajeput essential oil is about 0% to about 30%; ginger essential oil is about 0% to about 20%; jasmine essential oil is about 0% to about 30%; lemon basil essential oil is about 0% to about 20%; lavender essential oil is about 0% to about 20%; perilla essential oil is about 0% to about 30%; oregano essential oil is about 0% to about 30%; rose essential oil is about 0% to about 30%; maychang essential oil is about 0% to about 20%; pomu essential oil is about 0% to about 30%; eucalyptus essential oil is about 0% to about 20%; clove essential oil is about 0% to about 20%; turmeric essential oil is about 0% to about 30%; green tea essential oil is about 0% to about 30%; ylang ylang essential oil is about 0% to about 30%; sarsi essential oils is about 0% to about 30%; eucalyptus essential oils is about 0% to about 20%; and Cupressus funebris essential oils is about 0% to about 10%.

In the second aspect, the present invention provides a composition comprising natural essential oils as mentioned above, wherein in order to facilitate the adjustment of the concentration of the mixture of essential oils to suit the required needs, the composition further comprises carrier oil, solvent with appropriate proportion. In one preferred embodiment, said carrier oil is selected from at least one of the group consisting of coconut oil, argan oil, olive oil, almond oil, jojoba oil, vanilla oil, neem oil each is about 0% to about 70%; ethylic alcohol is about 0% to about 20 and water is about 0% to about 15%. Preferably, the ingredient carrier oils have content ratios as follows: coconut oil, argan oil, olive oil, almond oil, jojoba oil, vanilla oil, neem oil each is about 0% to about 50%; ethylic alcohol is about 0% to about 10 and water is about 0% to about 10%. Most preferably, these ingredient carrier oils have the following ratios: coconut oil, argan oil, olive oil, almond oil, jojoba oil, vanilla oil, neem oil, each of about 0% to about 30%; ethylic alcohol of about 0% to about 8%, and water of about 0% to about 8%.

The composition of the present invention is prepared in the usual method, by mixing the essential oil ingredients in the proportions as mentioned above (to obtain the natural essential oil composition of the present invention) and mixing with the carrier oils in the proportions as mentioned above (to obtain the composition of the present invention). The mixing temperature is not over 25° C.

### EXAMPLES

### Formulation Examples

Below are illustratable formulations of the natural essential oil composition t [symbol] nhiên of the present invention. These illustratable formulations are intended to help better understanding the invention but not to limit it in any way.

| | | Formulation | Formulation | Formulation | Formulation | Formulation |
|---|---|---|---|---|---|---|
| No. | Ingredient | 1 | 2 | 3 | 4 | 5 |
| 1 | Holy basil essential oil | 50 | 10 | 5 | 5 | 3 |
| 2 | Lemongrass essential oil | 15 | 10 | 5 | 5 | 2 |
| 3 | Patchouli essential oil | 15 | 30 | 10 | 5 | 3 |
| 4 | Essential oil of old coriander seeds | 10 | 10 | 5 | 5 | 2 |
| 5 | Essential oil of tangerine peels | 10 | 10 | 5 | 5 | 3 |
| 6 | Tea tree essential oil | 0 | 10 | 0 | 0 | 0 |
| 7 | Essential oil of grapefruit peels | 0 | 10 | 0 | 0 | 0 |
| 8 | Essential oil of orange peels | 0 | 10 | 0 | 0 | 0 |
| 9 | Frankincense essential oil | 0 | 0 | 0 | 5 | 2 |
| 10 | Mint essential oil | 0 | 0 | 10 | 5 | 3 |
| 11 | Garlic essential oil | 0 | 0 | 10 | 0 | 0 |
| 12 | Anise essential oil | 0 | 0 | 5 | 5 | 2 |
| 13 | Mugwort essential oil | 0 | 0 | 5 | 5 | 3 |
| 14 | Dill essential oil | 0 | 0 | 5 | 5 | 2 |
| 15 | Camphor essential oil | 0 | 0 | 5 | 3 | 3 |
| 16 | Cajeput essential oil | 0 | 0 | 0 | 3 | 2 |
| 17 | Ginger essential oil | 0 | 0 | 0 | 3 | 1 |
| 18 | Jasmine essential oil | 0 | 0 | 0 | 3 | 1 |
| 19 | Lemon basil essential oil | 0 | 0 | 10 | 3 | 1 |
| 20 | Lavender essential oil | 0 | 0 | 10 | 3 | 1 |
| 21 | Perilla essential oil | 0 | 0 | 5 | 3 | 1 |
| 22 | Oregano essential oil | 0 | 0 | 5 | 3 | 1 |
| 23 | Rose essential oil | 0 | 0 | 0 | 3 | 1 |
| 24 | Maychang essential oil | 0 | 0 | 0 | 3 | 1 |
| 25 | Pomu essential oil | 0 | 0 | 0 | 3 | 1 |
| 26 | Eucalyptus essential oil | 0 | 0 | 0 | 3 | 1 |
| 27 | Clove essential oil | 0 | 0 | 0 | 2 | 1 |
| 28 | Turmeric essential oil | 0 | 0 | 0 | 2 | 1 |
| 29 | Green tea essential oil | 0 | — | 0 | 2 | 1 |
| 30 | Ylang ylang essential oil | 0 | 0 | 0 | 2 | 1 |
| 31 | Sarsi essential oils | 0 | 0 | 0 | 2 | 1 |
| 32 | Eucalyptus essential oils | 0 | 0 | 0 | 2 | 1 |
| 33 | Cupressus funebris essential oils | 0 | 0 | 0 | 2 | 1 |
| 34 | Coconut oil | 0 | 0 | 0 | 0 | 10 |
| 35 | Argan oil | 0 | 0 | 0 | 0 | 10 |
| 36 | Olive oil | 0 | 0 | 0 | 0 | 10 |
| 37 | Almond oil | 0 | 0 | 0 | 0 | 5 |
| 38 | Jojoba oil | 0 | 0 | 0 | 0 | 5 |
| 39 | Vanilla oil | 0 | 0 | 0 | 0 | 3 |
| 40 | Neem oil | 0 | 0 | 0 | 0 | 2 |
| 41 | Ethyl alcohol | 0 | 0 | 0 | 0 | 5 |
| 42 | Water | 0 | 0 | 0 | 0 | 3 |
| | Total | 100 | 100 | 100 | 100 | 100 |

### Treatment Examples

Each formulation of the present invention was evaluated in 30 volunteers who have problems with sleep disorders (not deeply sleeping, difficulty in falling asleep).

Criteria for choosing volunteers: Men and women having problems with sleep disorders, agreeing to participate in the research, and in normal health status.

Exclusion criteria: Men and women having been using sedatives or medicine to treat insomnia.

Study methodology: Self-comparative clinical trials before and after using the composition of the present invention.

Test regimen: The natural essential oil composition is daily used before going to bed by spraying on pillows, bed sheets and soles for 1 continuous month.

Method for data collection: The volunteers were asked to fill their test results in same deceleration forms after each test.

The study result were evaluated as follows: Evaluation by the voluntary users on the following criteria: reducing stress, relaxation; effects of easy falling asleep; deeply sleeping; preventing cold and flu, respiratory diseases. All voluntary users were monitored and recorded all side effects: burning sensation, irritation, itching, allergies, etc.

Data processing method: The data were collected and processed according to medical statistical methods using software IPS 10.0.

Study conduct: The study was aimed to evaluate the effectiveness of the natural essential oil composition, without any other purposes. The participants of the study were volunteers whose personal information were kept confidential, only for study purposes, but not disclosed to any other third parties.

The study results are shown as follows:

Some traits of the voluntary users:

Distribution by age (n = 30)

| Age group | Amount (n) | % |
|---|---|---|
| ≤19 | 6 | 20.0 |
| 20-29 | 5 | 16.7 |
| 30-39 | 7 | 23.3 |
| 40-49 | 4 | 13.3 |
| 50-59 | 4 | 13.3 |
| ≥60 | 4 | 13.3 |
| Total | 30 | 100.0 |

Distribution by gender (n = 30)

| Gender | Amount (n) | % |
|---|---|---|
| Men | 19 | 63.3 |
| Women | 11 | 36.7 |
| Total | 30 | 100.0 |

Distribution by career (n = 30)

| Career | Amount (n) | % |
|---|---|---|
| Student | 13 | 41.7 |
| Staff | 8 | 26.7 |
| Self-employed | 9 | 31.7 |
| Total | 30 | 100.0 |

Symptoms (n = 30)

| Symptoms | Amount (n) | % |
|---|---|---|
| Insomnia, difficulty in falling asleep | 30 | 100 |
| Not deeply sleeping | 18 | 60 |
| Having respiratory problems (shortness | 16 | 53.3 |
| of breath, stuffy nose, allergic rhinitis or | | |
| rhinitis by air conditioner, snoring, etc) | | |
| Living often under stress | 22 | 73.33 |

After using the natural essential oil composition of the present invention according to the instructions for use, the results were recorded as follows:

Effects of reducing insomnia, sleeping disorders: reducing from 30 volunteers (100%) of symptoms of insomnia or difficulty sleeping before the treatment to 6 (20%) after the treatment, and 18 volunteers (60%) of not deeply sleeping before the treatment reduced to 3 (10%) after the treatment.

Effects of reducing nervous tension: after the treatment 100% of the volunteers feel comfortable, relax, reducing fatigue after a working day.

Effects of reducing headache symptom: reducing from 30 volunteers (100%) of headache symptom caused by insomnia before the treatment to 4 (13.4%) after the treatment

Effects of reducing dizziness symptom: reducing from 30 volunteers (100%) having dizziness symptom to 6 (20%).

Effects of reducing attention disorders: Attention disorders were studied such as difficulty in drawing attention from one thing to another, mental retardation, slowly accepting stimulation, or often forgetting. Before the treatment, 10 persons (33.3%) had symptoms of often forgetting, 8 persons (26.7%) had symptoms of attention disorders. After the treatment, these numbers has been reduced to 2 person (6.7%) and 3 persons (10%), accordingly.

Effects on respiratory tract, preventing respiratory problems and flu: Respiratory diseases were studied as such shortness of breath, stuffy nose, allergic rhinitis or rhinitis by air conditioner, snoring. Before the treatment, 16 persons (53.3%) had problems of their upper respiratory tracks. After the treatment, the number has been reduced to 6 persons (20%).

Effects of preventing flu: Before the treatment, 6 persons (20%) had symptoms of cold hands and feet, neck pain by cold. After the treatment it reduces to 2 persons (6.7%), 20 of 30 volunteers (66.7%) feel their bodies became warmer that prevent them from cold caused by either weather or air conditioners, 80% of the volunteers feel muscle relaxation after a hard working day.

Side effects: No case of burning sensation, irritation, itching, allergies was recorded when using the composition of the present invention.

Achieved Efficiency of the Present Invention

The natural essential oil composition of the present invention has the following advantages:
Making the users the feelings of relaxing nerves, sleeping well, preventing cold and flu, anti-fatigue, deodorization, enhancing immunity, stimulating hair growth, treating acne, whitening skin; supporting treatment of respiratory diseases, reducing pain, stimulating digestion, blood circulation, treating coughs; fixing aroma, reducing nasal congestion, treating joint pains, strengthening muscles, preventing hyperemia, preventing oxidation, reducing blood pressure, especially increasing sexual ability; etc.
Easy in preparing the composition, flexible in adjusting its ingredients upon purposes of use.
Simple to use, just by spraying the composition toward the user's skin, things or aira round, or applying on the skin.
Raw materials for preparing the composition are abundant and available.

1. A natural essential oil composition for relaxing nerves, sleeping well and preventing cold and flu comprising with the amounts of percentage by weight (wt %): holy basil essential oil is about 0.1% to about 60%; lemongrass essential oil is about 0.1% to about 55; patchouli essential oil is about 0.1% to about 30%; essential oil of old coriander seeds is about 0.1 to about 50% and essential oil of tangerine peels is about 0.1% to about 50%.

2. The natural essential oil composition of claim 1, wherein the amount (wt %) of holy basil essential oil is about 0.1% to about 45; the amount (wt %) of lemongrass essential oil is about 0.1% to about 30%; the amount (wt %) of patchouli essential oil is about 0.1% to about 20%; the amount (wt %) of essential oil of old coriander seeds is about 0.1 to about 40% and the amount (wt %) of essential oil of tangerine peels is about 0.1% to about 30%.

3. The natural essential oil composition of claim 1, wherein the amount (wt %) of holy basil essential oil is about 0.1% to about 30%; lemongrass essential oil is about 0.1% to about 25%; patchouli essential oil is about 0.1% to about 15; essential oil of old coriander seeds is about 0.1 to about 30% and essential oil of tangerine peels is about 0.1% to about 25%.

4. The natural essential oil composition of claim 1, wherein the composition further comprises tea tree essential oil; essential oil of grapefruit peels; and essential oil of orange peels.

5. The natural essential oil composition of claim 4, wherein the amount (wt %) of tea tree essential oil is about 0% to about 60%; the amount (wt %) of essential oil of grapefruit peels is about 0% to about 50%; and the amount (wt %) of essential oil of orange peels is about 0% to about 50.

6. The natural essential oil composition of claim 5, wherein the amount (wt %) of tea tree essential oil is about 0% to about 40%; essential oil of grapefruit peels is about 0% to about 35; and essential oil of orange peels is about 0% to about 30%.

7. The natural essential oil composition of claim 5, wherein the amount (wt %) of tea tree essential oil is about 0% to about 30%; essential oil of grapefruit peels is about 0% to about 20%; and essential oil of orange peels is about 0% to about 25%.

8. The natural essential oil composition of claim 5, wherein the composition further comprises with the amounts of percentage by weight (wt %): mint essential oil is about 0% to about 50%; garlic essential oil is about 0% to about 30%; anise essential oil is about 0% to about 40%; mugwort essential oil is about 0% to about 50%; dill essential oil is about 0% to about 50%; camphor essential oil is about 0% to about 30%; basil essential oil is about 0% to about 30%; lavender essential oil is about 0% to about 30%; perilla essential oil is about 0% to about 50%; and oregano essential oil is about 0% to about 50.

9. The natural essential oil composition of claim 6, wherein the composition further comprises with the amounts by weight: mint essential oil is about 0% to about 40%; garlic essential oil is about 0% to about 20%; anise essential oil is about 0% to about 25%; mugwort essential oil is about 0% to about 40%; dill essential oil is about 0% to about 40%; camphor essential oil is about 0% to about 25%; basil essential oil is about 0% to about 25%; lavender essential oil is about 0% to about 25%; perilla essential oil is about 0% to about 40%; and oregano essential oil is about 0% to about 40.

10. The natural essential oil composition of claim 7, wherein the composition further comprises with the amounts by weight: mint essential oil is about 0% to about 30%; garlic essential oil is about 0% to about 10%; anise essential oil is about 0% to about 15; mugwort essential oil is about 0% to about 30%; dill essential oil is about 0% to about 30%; camphor essential oil is about 0% to about 20%; basil essential oil is about 0% to about 20%; lavender essential oil is about 0% to about 20%; perilla essential oil is about 0% to about 30%; and oregano essential oil is about 0% to about 30%.

11. The natural essential oil composition of claim 8, wherein the composition further comprises with the amounts by weight: frankincense essential oil is about 0% to about 20%; mint essential oil is about 0% to about 50%; garlic essential oil is about 0% to about 30%; anise essential oil is about 0% to about 40%; mugwort essential oil is about 0% to about 50%; dill essential oil is about 0% to about 50%; camphor essential oil is about 0% to about 30%; cajeput essential oil is about 0% to about 50%; ginger essential oil is about 0% to about 40%; jasmine essential oil is about 0% to about 50%; lemon basil essential oil is about 0% to about 30%; lavender essential oil is about 0% to about 30%; perilla essential oil is about 0% to about 50%; oregano essential oil is about 0% to about 50%; rose essential oil is about 0% to about 50%; maychang essential oil is about 0% to about 40%; pomu essential oil is about 0% to about 50%; eucalyptus essential oil is about 0% to about 30%; clove essential oil is about 0% to about 40%; turmeric essential oil is about 0% to about 50%; green tea essential oil is about 0% to about 50%; ylang ylang essential oil is about 0% to about 50%; sarsi essential oils is about 0% to about 50%; eucalyptus essential oils is about 0% to about 30%; and Cupressus funebris essential oils is about 0% to about 20%.

12. The natural essential oil composition of claim 9, wherein the composition further comprises with the amounts by weight: frankincense essential oil is about 0% to about 15; mint essential oil is about 0% to about 40%; garlic essential oil is about 0% to about 20%; anise essential oil is about 0% to about 25%; mugwort essential oil is about 0% to about 40%; dill essential oil is about 0% to about 40%; camphor essential oil is about 0% to about 25%; cajeput essential oil is about 0% to about 40%; ginger essential oil is about 0% to about 30%; jasmine essential oil is about 0% to about 40%; lemon basil essential oil is about 0% to about 25%; lavender essential oil is about 0% to about 25%; perilla essential oil is about 0% to about 40%; oregano essential oil is about 0% to about 40%; rose essential oil is about 0% to about 40%; maychang essential oil is about 0% to about 30%; pomu essential oil is about 0% to about 40%; eucalyptus essential oil is about 0% to about 25%; clove essential oil is about 0% to about 30%; turmeric essential oil is about 0% to about 40%; green tea essential oil is about 0% to about 40%; ylang ylang essential oil is about 0% to about 40%; sarsi essential oils is about 0% to about 40%; eucalyptus essential oils is about 0% to about 25%; and Cupressus funebris essential oils is about 0% to about i 5%.

13. The natural essential oil composition of claim 10, wherein the composition further comprises with the amounts by weight: frankincense essential oil is about 0% to about 10%; mint essential oil is about 0% to about 30%; garlic essential oil is about 0% to about 10%; anise essential oil is about 0% to about 15; mugwort essential oil is about 0% to about 30%; dill essential oil is about 0% to about 30%; camphor essential oil is about 0% to about 20%; cajeput essential oil is about 0% to about 30%; ginger essential oil is about 0% to about 20%; jasmine essential oil is about 0% to about 30%; lemon basil essential oil is about 0% to about 20%; lavender essential oil is about 0% to about 20%; perilla essential oil is about 0% to about 30%; oregano essential oil is about 0% to about 30%; rose essential oil is about 0% to about 30%; maychang essential oil is about 0% to about 20%; pomu essential oil is about 0% to about 30%; eucalyptus essential oil is about 0% to about 20%; clove essential oil is about 0% to about 20%; turmeric essential oil is about 0% to about 30%; green tea essential oil is about 0% to about 30%; ylang ylang essential oil is about 0% to about 30%; sarsi essential oils is about 0% to about 30%; eucalyptus essential oils is about 0% to about 20%; and Cupressus funebris essential oils is about 0% to about 10%.

14. The natural essential oil composition of claim 11, wherein the composition further comprises carrier oil, solvent with the amounts by weight: coconut oil, argan oil, olive oil, almond oil, jojoba oil, vanilla oil, neem oil each is about 0% to about 70%; ethylic alcohol is about 0% to about 20 and water is about 0% to about 15%.

16. The natural essential oil composition of claim 14. wherein the amounts by weight of coconut oil, argan oil, olive oil, almond oil, jojoba oil, vanilla oil, neem oil each is about 0% to about 50%; the amounts by weight of ethylic alcohol is about 0% to about 10 and the amounts by weight of water is about 0% to about 10%.

17. The natural essential oil composition of claim 14, wherein the amounts by weight of coconut oil, argan oil, olive oil, almond oil, jojoba oil, vanilla oil, neem oil each is about 0% to about 30%; the amounts by weight of ethylic alcohol is about 0% to about 8 and the amounts by weight of water is about 0% to about 8%.
